(19) United States
(12) Patent Application Publication
(10) Pub. No.: US 2018/0071524 A1
(43) Pub. Date: Mar. 15, 2018

(54) DEVICE, METHOD AND COMPOUND FOR THE BONE RECONSTRUCTION OF A VERTEBRAE

(71) Applicant: GEOCORAIL, Marseille (FR)

(72) Inventors: DOMINIQUE MICHEL, Paris (FR); Philippe POULETTY, Paris (FR); Pierre-Joseph SCHARR, Paris (FR)

(73) Assignee: GEOCORAIL, Marseille (FR)

(21) Appl. No.: 15/564,437

(22) PCT Filed: Apr. 1, 2016
(86) PCT No.: PCT/FR2016/050749

§ 371 Date: Oct. 5, 2017

(30) Foreign Application Priority Data
Apr. 7, 2015 (FR) 1552970

(51) Int. Cl. A61N 1/20; A61N 1/32; A61N 1/378

(52) U.S. Cl. CPC A61N 1/205; A61N 1/0416; A61N 1/378; A61N 1/326

(57) ABSTRACT

A device (1) for bone reconstruction, has an anode (A), a cathode (C), an electrical circuit (2) to which the cathode (C) and anode (A) are connected, and an electric generator (G), preferably a current generator, for supplying power to this circuit; the device being suitable for implantation within the body of a vertebrate.

The present invention primarily relates to a system and a method for bone reconstruction.

Bone reconstruction is generally slow.

A particular object of the invention is to provide a device and a method allowing defective cartilage of bony portions of the body of a vertebrate to be reconstructed, in an accelerated manner.

According to a first subject of the invention, a device for bone reconstruction is characterized in that it comprises:
an anode;
a cathode;
an electrical circuit to which the cathode and the anode are connected;
an electric generator, preferably a current generator, for supplying power to the electrical circuit;
this device being suitable for implantation within the body of a vertebrate.

The anode may be a titanium needle. The cathode is advantageously made of a conductive metal that is non-toxic for the body. The generator comprises a miniaturized battery.

Advantageously, the device comprises means for cyclically varying the intensity of the current in the circuit, preferably between values corresponding alternately to the phase of precipitation/deposition of Ca⁺⁺ and of Mg⁺⁺ in optimum amounts and to the phase in which the redilution of an excess fraction of the previously deposited magnesium is favored, until reaching, in the conglomerate thus formed, a Ca/Mg ratio corresponding to a desired degree of hardness and/or of roughness.

The cathode may comprise a calcium-magnesium coating.

According to a second subject of the invention, a method for bone reconstruction in a vertebrate provided with a device according to the invention is characterized in that it comprises a step of making a current flow through the circuit of the device.

Advantageously, the intensity of the current in the circuit is varied cyclically, preferably between values corresponding alternately to the phase of precipitation/deposition of Ca⁺⁺ and of Mg⁺⁺ in optimum amounts and to the phase in which the redilution of an excess fraction of the previously deposited magnesium is favored, until reaching, in the conglomerate thus formed, a Ca/Mg ratio corresponding to a desired degree of hardness and/or of roughness.

Preferably, such a method comprises a step of providing a medicinal compound suitable for the gradual, and preferably slow, diffusion of calcium and/or magnesium salts into the blood of the vertebrate.

According to a third subject of the invention, a compound employed in a method according to the invention comprises calcium salts and/or magnesium salts. It may be provided in an injectable form, in a drinkable form or else in the form of a pill or of a capsule.

Several embodiments of the invention will be described below, by way of non-limiting examples, with reference to the appended drawings, in which:

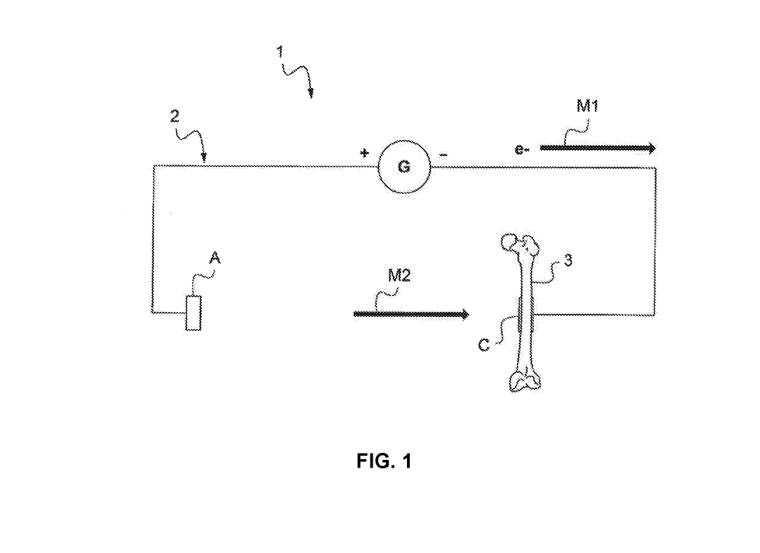

FIG. 1 is a schematic view illustrating a device and a method according to the invention.

It is known, in particular from document WO 2005/047571, that the metal structure forming the cathode portion of an electrolysis system in contact with a marine or brackish aqueous medium is coated with salt deposits, referred to by specialists as “calcium-magnesium” deposits, caused by the precipitation of compounds, in particular CaCO₃ (calcium carbonate) and Mg(OH)₂ (magnesium hydroxide), onto this structure due to ions arising from elements (Ca, Mg) and gases (CO₂, O₂) dissolved in an electrolytic medium (seawater, conductive soil or in the brackish water of lagoons) combining. The system according to the invention uses this principle and develops a bone reconstruction process, in particular a method for reconstructing defective cartilage of bony portions of the body of a vertebrate.

As illustrated in FIG. 1, a device 1 according to the invention comprises in particular an anode A, a cathode C and an electric generator G together forming an open electrical circuit 2 between the anode A and the cathode C. The device 1 is suitable for implantation within the body. The generator is advantageously a miniaturized battery. The anode A is preferably a titanium needle. The cathode is made of a conductive metal that is non-toxic for the body.

With the cathode C being brought into contact with the portion of a bone 3 to be regenerated and the anode being brought into proximity thereto, a method according to the invention, implementing the device 1, consists in making a low-intensity current M1 flow through the circuit 2. This results in a migration M2 of magnesium and calcium ions, and of other materials contained in the blood, in the direction of the cathode C. This results in the defective bony portions being filled in and/or reinforced.

The invention also comprises a medicinal compound allowing the slow, gradual diffusion of calcium and/or magnesium salts. Specifically, for greater efficacy, the method advantageously comprises the administration of such a compound. The compound may be envisaged for oral administration; however, other routes may be used, in particular directly into the bone.

Surprisingly, it appears that the method according to the invention is more effective when the intensity of the current by the generator G is varied cyclically between end values corresponding alternately to the phase of precipitation/deposition of Ca⁺⁺ and of Mg⁺⁺ in optimum amounts and to the phase in which the redilution of an excess fraction of the previously deposited magnesium is favored, until reaching, in the conglomerate thus formed, a Ca/Mg ratio corresponding to a desired degree of hardness and/or of roughness.

Preferably, the cathode C comprises a coating produced in the laboratory. The coating is produced by means of a method that allows the bone reconstruction process to be initiated, i.e. the formation of a deposit similar to that which would be obtained when the cathode is implanted. Such a method comprises immerging the cathode in an ionic solution comprising a mixture of ions suitable for producing a deposit similar to that obtained in the human body, and subjecting the cathode to a current such that some of these ions are deposited thereon. Once the cathode is implanted, the reconstruction process is continued in vivo.

Of course, the invention is not limited to the examples described above.

1. A device (1) for bone reconstruction, characterized in that it comprises:
an anode (A);
a cathode (C);
an electrical circuit (2), to which said cathode (C) and said anode (A) are connected; and
an electric generator (G), preferably a current generator, for supplying power to said electrical circuit;
said device being suitable for implantation within the body of a vertebrate.

2. The device as claimed in claim 1, characterized in that the anode is a titanium needle.

3. The device as claimed in claim 1, characterized in that the cathode is made of a conductive metal that is non-toxic for the body.

4. The device as claimed in claim 1, characterized in that the generator (G) comprises a miniaturized battery.

5. The device as claimed in claim 1, characterized in that it comprises means for cyclically varying the intensity of the current in the circuit, preferably between values corresponding alternately to the phase of precipitation/deposition of Ca++ and of Mg++ in optimum amounts and to the phase in which the redilution of an excess fraction of the previously deposited magnesium is favored, until reaching, in the conglomerate thus formed, a Ca/Mg ratio corresponding to a desired degree of hardness and/or of roughness.

6. The device as claimed in claim 1, characterized in that the cathode comprises a calcium-magnesium coating.

7. A medicinal compound for bone reconstruction in a vertebrate provided with a device (1) as claimed in claim 1, characterized in that it comprises calcium salts and/or magnesium salts.

8. The compound as claimed in claim 7, characterized in that it is provided in an injectable form.

9. The compound as claimed in claim 7, characterized in that it is provided in a drinkable form.

10. The compound as claimed in claim 7, characterized in that it is provided in the form of a pill or of a capsule.
